(19) United States
(12) Patent Application Publication
(10) Pub. No.: US 2017/0136076 A1
(43) Pub. Date: May 18, 2017

(54) THERAPEUTIC COMPOSITIONS COMPRISING HERBAL EXTRACTS AND ESSENTIAL OILS FOR SMOKING AND VAPORIZATION

(71) Applicant: NISARGA BIOTECH PRIVATE LIMITED, Satar (IN)

(72) Inventors: Girish Sudhakar SOMAN, Satar (IN); Shrikrishna Govind PHADKE, Satar (IN)

(21) Appl. No.: 15/343,022

(22) Filed: Nov. 3, 2016

Related U.S. Application Data
(63) Continuation of application No. PCT/IN2016/050374, filed on Oct. 28, 2016.
(60) Provisional application No. 62/250,287, filed on Nov. 3, 2015.

(30) Foreign Application Priority Data
Oct. 30, 2015 (IN) 4124/MUM/2015

(51) Int. Cl. A61K 36/185; A61K 9/00; A61K 36/68; A61K 36/8905; A61K 36/23; A61K 36/84; A61K 36/53; A61K 36/54; A61K 36/906; A61K 36/835; A61K 36/88; A61K 36/81; A61K 36/48; A61K 36/9068; A61K 36/328; A61K 36/9066; A61K 36/77; A61K 36/28; A61K 31/05; A61K 31/352; A61K 36/235; A61K 36/67; A61K 36/534; A61K 36/484; A61K 36/59; A61K 47/44

(52) U.S. Cl. CPC A61K 36/185; A61K 47/44; A61K 9/007; A61K 36/68; A61K 36/8905; A61K 36/23; A61K 36/84; A61K 36/53; A61K 36/54; A61K 36/906; A61K 36/835; A61K 36/88; A61K 36/81; A61K 36/48; A61K 36/9068; A61K 36/328; A61K 36/9066; A61K 36/77; A61K 36/28; A61K 31/05; A61K 31/352; A61K 36/235; A61K 36/67; A61K 36/534; A61K 36/484; A61K 36/59; A61K 2236/37

(57) ABSTRACT

The present invention discloses therapeutic compositions comprising herbal extracts and essential oils for smoking and vaporization useful in the treatment of various symptoms and diseases. More particularly the invention relates to compositions comprising herbal extracts and essential oils capable of providing therapeutic benefits, which helps to improve conditions like cough, cold, pain, energy, relaxation, calmness, digestion, immune system disorders and neurological disorders etc. The invention further relates to methods for treating the same using the compositions of the invention. The invention also discloses method of formulating a blend of herbal CO₂ extracts and essential oils without the use of any type of synthetic carriers or residual solvent or synthetic chemicals, thus improving quality, safety and efficacy.

### CROSS-REFERENCE TO RELATED APPLICATIONS

This application claims priority to a U.S. Provisional Application No. 62/250,287 filed on Nov. 3, 2015. This application claims priority to a PCT International Application No.: PCT/IN2016/050374 filed on Oct. 28, 2016. The entire disclosure of the prior application is hereby incorporated by reference herein in its entirety.

### TECHNICAL FIELD

The present invention relates to therapeutic compositions comprising herbal extracts and essential oils for smoking and vaporization useful in the treatment of various symptoms and diseases. More particularly the invention relates to compositions comprising herbal extracts and essential oils capable of providing therapeutic benefits, which helps to improve conditions like cough, cold, pain, energy, relaxation, calmness, digestion, immune system disorders and neurological disorders etc. The invention further relates to methods for treating the same using the compositions of the invention. The invention also relates to formulating a blend of herbal CO₂ extracts and essential oils without the use of any type of synthetic carrier or residual solvent or synthetic chemicals, thus improving quality, safety and efficacy of the said therapeutic composition.

### BACKGROUND AND PRIOR ART

According to “Ashtanghridaya” following herbs were used in “Dhumpan” (meaning medicated smoke inhalation) which includes Nardostachys jatamansi, Cinnamomum zeylanicum, Boswellia serrata, Ricinus communis, Cocos nucifera, Acacia catechu, Aquilaria agallocha, cyperus rotundus, Commiphora mukul, Achyranthes aspera, Cedrus deodara, Parmelia perlata, Coleus zeylanicus, Vetiveria zizaioides, Murraya paniculata, Aegle marmalos, Celastru paniculata, Curcuma longa, Zingiber officinalis, Ceiba pentandra, Emblica officinalis, Dashmool, Triphala etc. and importance of the Dhumpan is also quoted in it.

Dhum means smoke and pan means to ingest or inhale. According to Ayurveda, Dhumpanam or Dhumpan simply means medicated or medicine or medical smoking. It is totally different from cigarette smoking. It is the use of fire for burning the medicinal herb in treatment and also the journey of medicines on a vehicle of smoke. Medicines can reach the minute cavities quickly and can act quickly. It is a therapy used in treatment of various disorders. It is mainly used in treatment of vata and kapha dominated diseases. It is also indicated in day to day life activity for normal healthy people to achieve its benefits. Medicinal or Medical smoking (Dhum Pan) is beneficial in destroying antigens present in the environment, enhances activity of sense organs, gives mental relaxation, acts as a mouth freshener and useful in hair fall. Specific type of Dhumpan is used in treatment of diseases like cough, asthma, coryza, excess sleep, sleeplessness and many other ailments originating above the shoulder.

A vaporizer or vaporiser is a device used to vaporize the active ingredients of plant material, commonly cannabis, tobacco, or other herbs or blends for the purpose of inhalation. However, they can be used with pure chemicals when mixed with plant material (e.g. tobacco-free nicotine). The proposed factors affecting output include temperature, specimen density, weight and content of water, essential oils and consistency of material in the filling chamber, storage time of the vapor, inhalation method.

Vaporizers contain various forms of extraction chambers including straight bore, venturi or sequential venturi and are made of materials such as porcelain, plastic, metal or glass. The extracted vapor may be collected in an inflatable bag or inhaled directly through a hose or pipe. With no combustion happening when used properly and appropriate temperatures, a significantly better efficiency in vaporising the ingredients can be obtained. Hence, the irritating and harmful effects of smoking are heavily reduced as in first or second hand smoke.

An electronic cigarette (e-cig or e-cigarette), personal vaporizer (PV) or electronic nicotine delivery system (ENDS) is a battery-powered vaporizer that feels similar to tobacco smoking. The user inhales an aerosol commonly called vapor, rather than cigarette smoke. E-cigarettes typically have a heating element that atomizes a liquid solution known as e-liquid. There are disposable or reusable versions. E-liquids usually contain a mixture of propylene glycol, glycerin, nicotine, and flavorings. E-liquids are also available without propylene glycol, without nicotine, or without flavors.

The benefits and health risks of electronic cigarettes are uncertain. One review found limited evidence of a benefit as a smoking cessation aid but there is no evidence that they are better than regulated medication for quitting smoking. Their usefulness in tobacco harm reduction is unclear. One other review found their safety risk is similar to that of smokeless tobacco. Another review describes that the United States Food and Drug Administration (FDA)-approved products, such as nicotine inhalers, are probably safer than e-cigarettes.

Vaporizers are also used to inhale marijuana. Of the studies about vaporizing marijuana, few have addressed the quality of the vapor extracted and delivered; instead, studies usually focus on the mode of usage of the vaporizers. There are 483 identifiable chemical constituents known to exist in the cannabis plant, and at least 85 different cannabinoids have been isolated from the plant. The aromatic terpenoids begin to vaporize at 126.0° C. (258.8° F.), but the more bio-active tetrahydrocannabinol (THC), cannabidiol (CBD) and cannabinol (CBN) do not vaporize until near their respective boiling points: THC-157° C. (315° F.), CBD-160-180° C. (320° F.-356° F.), and CBN-185° C. (365° F.).

Studies have shown that vaporizing cannabis exposes the user to lower levels of harmful substances than smoking cannabis. These findings are important because it is estimated that 10-20 percent of patients with chronic pain, multiple sclerosis, epilepsy, and HIV/AIDS have admitted to smoking cannabis for therapeutic purposes. Another study found that smoking cannabis sativa reduced daily pain by 34% in patients, a statistically significant effect.

In a study published in the Journal of Psychopharmacology in May 2008, it was stated that vaporizers were a “suitable method for the administration of THC.” A 2007 study by University of California, San Francisco, published in the Journal of the American Academy of Neurology, found that “there was virtually no exposure to harmful combustion products using the vaporizing device.” A study performed by researchers at Leiden University in 2006 found that vaporizers were “safe and effective cannabinoid delivery system(s)”. This study stated that the amount of THC delivered by vaporizers were equivalent to the amount delivered by smoking. In the light of the above detailed studies & reports thereof, it appears that vaporizers are medically sound devices for delivering THC.

Research on the use of vaporizers found that the delivery efficiency is highest at around 226° C. (439° F.), falling to about half efficiency at 150° C. (302° F.) to 180° C. (356° F.) degrees depending on material. The purest preparations produced the highest efficiencies, about 56% for pure THC versus 29% for plant material (female flower tops) with 12% THCA content. Besides THC, several other cannabinoids as well as a range of other plant components including terpenoids were detected in the plant material. Using pure THC in the vaporizer, no degradation products (delta-8-THC (D8-THC), cannabinol (CBN), or unknown compounds) were detected by HPLC analysis. The longer vapor is stored, the more THC is lost as it condenses on the surface of the vaporizer or the balloon. This loss may be negligible over a few minutes but may exceed 50% after 90 minutes. The Leiden University study found that as much as 30%-40% of inhaled THC was not absorbed by the lungs but simply exhaled. However, they did not find large individual differences in the amounts exhaled.

Vaporizers are sometimes used by chefs as a method of applying controlled heat to herbs and spices to release flavors that are otherwise difficult to titrate or apply, or that might be spoiled by overheating during cooking. Grant Achatz, chef-proprietor of Alinea in Chicago, “uses the aroma-filled bags as place-mats, punctured when plates are placed in front of the customer”.

There is ample patented literature available on herbal and aromatherapy vaporizers.

US20090078253A1 provides an herbal and aromatherapy vaporizer, which is arranged to direct hot air flow moving longitudinally to retain hot air contacting the treatment element for a longer period of time and the hot air carries away more functional constituents from the treatment element to increase vapor concentration. The vaporizer includes a heat generator, a vapor outlet, a treatment crucible and an inhaler unit all aligned longitudinally. The air is heated in the heat generator and excited at the vapor outlet, and then longitudinally passes through the treatment element in the treatment crucible. The treatment element is heated to release functional constituents which are carried away by the hot air to subsequently pass through the inhaling guider at the exhausting end thereof. The vapor is collected by an inflatable pocket at the exhausting end and the user inhales the vapor at an opposed mouth piece end of the inhaling guider.

U.S. Pat. No. 8,869,792B1 describes a portable apparatus for vaporizing herbs and plant comprising housing or mixing chamber, which incorporates a vaporizing/heating element and rotatable mixing element that allows for an effective level of mixing of heated plants and the herbs during vaporization process.

U.S. Pat. No. 6,250,301B1 provides a vaporizer apparatus and method for uniformly extracting active ingredients of a specimen of a crude natural product or inert particulate matrix impregnated with volatile substance, without pyrolysis, which uses hot air or heated inert gas stream to volatize the specimen.

U.S. Pat. No. 3,949,743 provides improved method and apparatus for the treatment of respiratory, skin eye and related ailments and conditions wherein steam or a related vapor at a reduced temperature is admixed with particulate medicament and exposed to an afflicted area within a confirm space.

US20150125136A1 describes an appliance for volatizing active substances in either herbal material or fluid extract for inhalation, including a material vaporization surface with a plurality of convective air flow apertures, a conductive heater and convective heater.

US20110126831A1 discloses a portable essence vaporizer which comprises a heat source for heating up an air flow which is passed through the substance from which essence is to be extracted and supplied to the user, and it comprises heat exchanger which provides heat generated by the source to the air flow and the thermal insulation which insulates the exterior from the heat source and the exchanger, said insulation being preferably micro porous and having monolithic structure of metal oxide, amorphous silica or glass filament and heat exchanger comprising an open cell foam structure; the vaporizer provides a satisfactory extraction and at the same time is of reduced size and has reduced electrical consumption.

US20090302019A1 describes a volatile material vaporizing apparatus feature a heating element and heating element controls supported in separate housing to avoid exposure of the controls to heat produced by the heating element. Another embodiment features an elongate heating element supported entirely within a housing having an open end sized to fit over the bowl of existing smoking device so that drawing air through the smoking devices induces air flow into an inlet opening of the housing, along the heating element disposed therein and onward to the smoking device.

U.S. Pat. No. 6,513,524B1 describes process for inhalation and inhaler for production of aroma and active substance containing vapors of plant material and/or fluids via hot air.

Therefore, there remains a need in the art to provide herbal formulation for vaporization/smoking comprising safe and effective extracts and essential oils of two or more herbs, effective for the treatment of symptoms of various diseases and obtaining various types of therapeutic benefits without any side effects.

### OBJECTIVES OF THE INVENTION

The main objective of the present invention is to provide compositions comprising safe and effective herbal extracts and essential oils for smoking and vaporization, useful in the treatment of various symptoms, diseases and capable of providing therapeutic benefits, which helps to improve conditions like cough, cold, pain, energy depletion, improve relaxation, calmness, digestion, immune system disorders and neurological disorders etc.

Another objective is to provide a blend of 100% natural herbal extracts, and/or essential oils along with 100% natural fixed oils as a carrier and without using any synthetic carrier such as PEG, Glycerol and PG, for use in vaporizing devices such as E-cigarettes with batteries, dabbing devices such as bongs or nebulizing devices capable of producing vapors without smoke.

### SUMMARY OF THE INVENTION

Accordingly in one aspect the invention discloses therapeutic herbal composition comprising two or more effective CO₂ extracts and/or essential oils from the herbs selected from the group consisting of Cinnamomum zeylanicum (Dried bark), Elettatia cardamomum (Dried fruits and seeds), Aquilaria agallocha (Dark resinous heartwood), Crocus sativus (Stigmas of flower), Santalum album (Dried wood), Withania somnifera (Dried roots), Mucuna pruriens (Dried seeds), Celastrus paniculatus (Dried seeds), Zingiber officinale (Dried rhizomes), Emblica officinalis (Dried fruits), Terminalia chebula (Dried fruits), Terminalia bellerica (Dried fruits), Solanum xanthocarpum (Dried fruits), Glycyrrhiza glabra (Dried roots), Ocimum sanctum (Dried aerial part), Curcuma longa (Dried rhizomes), Apium graveolens (Dried seeds and roots), Mentha piperita (Dried leaves), Commiphora mukul (Gum), Cinnamomum camphora (Dried stem and leaves), Boswellia serrata (Gum Resins), Vitex negundo (Dried leaves), Bacopa monnieri (Aerial part), Cyperus rotundus (Dried rhizomes), Centella asiatica (Whole Plant), Myristica fragrans (Dried kernel of the seed), Valeriana wallichii (Dried roots), Nardostachys jatamansi (Dried fruits and seeds) used for vaporization/smoking. The origin of the plants used in formulations belongs to various parts of India.

The supercritical CO₂ extracts used in the present invention is obtained by supercritical CO₂ extraction technology with or without entrained ethanol, which does not leave any hazardous solvent residue. The temperature for extraction is between 31° C. to 50° C. and the separation of phytochemical is always at lower temperature than the extraction temperature i.e. 20° C.-35° C., therefore it ensures that retention of all temperature sensitive ingredients and integrity of extracted phytochemicals is also maintained. Such extracts are also combined with essential oils of above herbs obtained using steam distillation for adjusting the vaporization temperature of the blends and improving the efficacy of vapors and vaporization.

In another aspect, the invention discloses various combinations of above mentioned extracts in various levels of quantity to produce desired therapeutic or pharmacological effects. The various combinations are prepared by using different extracts and/or essential oils along with one or combination of fixed oils like flax seed oil, Soyabean oil, Seasame oil, Castor oil etc. as carrier oils instead of any other synthetic carriers for different disease conditions like cough, cold, pain, energy, relaxation, calmness, digestion, immune system disorder and neurological disorders etc.

In yet another aspect, due to this novel way of drug delivery of using vapors for the therapeutic benefit of different herbs mentioned above, the dosage required is much lower compared to oral dosage and the dosage form used in all earlier prior arts.

### DETAILED DESCRIPTION OF THE INVENTION

In accordance with the above aspects, the invention will now be described in connection with certain preferred and optional embodiment, so that various aspects thereof may be more fully understood and appreciated.

Source of Biological Material:

The herbs used in the present invention for the preparation of extracts are widely distributed throughout India. All the herbs mentioned in the specification are of Indian origin.

The present invention describes therapeutic herbal compositions for vaporization/smoking comprising mixture of extracts of two or more herbs selected from the group consisting of Cinnamomum zeylanicum (Dried bark), Elettaria cardamomum (Dried fruits and seeds), Aquilaria agallocha (Dark resinous heartwood), Crocus sativus (Stigmas of flower), Santalum album (Dried wood), Withania somnifera (Dried roots), Mucuna pruriens (Dried seeds), Celastrus paniculatus (Dried seeds), Zingiber officinale (Dried rhizomes), Emblica officinalis (Dried fruits), Terminalia chebula (Dried fruits), Terminalia bellerica (Dried fruits), Solanum xanthocarpum (Dried fruits), Glycyrrhiza glabra (Dried roots), Ocimum sanctum (Dried aerial part), Curcuma longa (Dried rhizomes), Apium graveolens (Dried seeds and roots), Mentha piperita (Dried leaves), Commiphora mukul (Gum), Cinnamomum camphora (Dried stem and leaves), Boswellia serrata (Gum Resins), Vitex negundo (Dried leaves), Bacopa monnieri (Aerial part), Cyperus rotundus (Dried rhizomes), Centella asiatica (Whole Plant), Myristica fragrans (Dried kernel of the seed), Valeriana Wallichii (Dried roots), Nardostachys jatamansi (Dried fruits and seeds useful in the treatment of various symptoms and getting different types of therapeutic benefits to the body. Such extracts are mixed together with steam distilled oils and/or CO₂ extracted oils from above herbs.

The herbs are extracted using supercritical CO₂ extraction technology with or without entrained ethanol, which does not leave any hazardous solvent residue. The temperature for extraction is between 31° C. to 50° C. and the separation of phytochemical is almost low temperature i.e. 20° C.-31° C., therefore it insures that retention of all temperature sensitive ingredients and integrity of phytochemicals is also maintained.

The inventors of present invention have screened many plants on the basis of text available in Ayurveda and other alternative medicine. After screening extensively depending upon the therapeutic and pharmacological action, different combinations of herbal extracts are selected in designing the specific compositions, for the treatment of specific ailments. Accordingly, the current invention comprises a mixture of extracts and/or essential oils of two or more herbs selected from the group consisting of Cinnamomum zeylanicum (Dried bark), Elettaria cardamomum (Dried fruits and seeds), Aquilaria agallocha (Dark resinous heartwood), Crocus sativus (Stigmas of flower), Santalum album (Dried wood), Withania somnifera (Dried roots), Mucuna pruriens (Dried seeds), Celastrus paniculatus (Dried seeds), Zingiber officinale (Dried rhizomes), Emblica officinalis (Dried fruits), Terminalia chebula (Dried fruits), Terminalia bellerica (Dried fruits), Solanum xanthocarpum (Dried fruits), Glycyrrhiza glabra (Dried roots), Ocimum sanctum (Dried aerial part), Curcuma longa (Dried rhizomes), Apium graveolens (Dried seeds and roots), Mentha piperita (Dried leaves), Commiphora mukul (Gum), Cinnamomum camphora (Dried stem and leaves), Boswellia serrata (Gum Resins), Vitex negundo (Dried leaves), Bacopa monnieri (Aerial part), cyperus rotundus (Dried rhizomes), Centella asiatica (Whole Plant), Myristica fragrans (Dried kernel of the seed), Valeriana Wallichii (Dried roots), Nardostachys jatamansi (Dried fruits and seeds), in association with fixed oils.

Accordingly, in preferred embodiment, the therapeutic herbal formulations comprising vaporizing blends of two or more of Cinnamomum zeylanicum CO₂ oil in an amount 0.1 to 25% of the total formulation, Elettaria cardamomum oil in an amount 0.1 to 53% of the total formulation, Aquilaria agallocha CO₂ extract in an amount 0.1 to 1% of the total formulation, Crocus sativus oil in an amount 0.1 to 11% of the total formulation, Santalum album steam distilled oil in an amount 0.1 to 20% of the total formulation, Withania somnifera CO₂ Extract in an amount 0.1 to 5% of the total formulation, Mucuna pruriens CO₂ Extract in an amount 0.1 to 5% of the total formulation, Emblica officinalis CO₂ Extract in an amount 0.1 to 2.0% of the total formulation, Terminalia chebula CO₂ Extract in an amount 0.1 to 2.0% of the total formulation, Terminalia bellerica CO₂ Extract in an amount 0.1 to 2.0% of the total formulation, Solanum xanthocarpum CO₂ Extract in an amount 0.1 to 1% of the total formulation, Glycyrrhiza glabra CO₂ Extract in an amount 0.1 to 1% of the total formulation, Ocimum sanctum CO₂ oil/steam distilled oil in an amount 0.1 to 35% of the total formulation, Curcuma longa CO₂ oil in an amount 0.1 to 30% of the total formulation, Zingiber officinale CO₂ oil/steam distilled oil in an amount 0.1 to 20% of the total formulation, Apium graveolens CO₂ oil in an amount 0.1 to 20% of the total formulation, Mentha piperita oil in an amount 0.1 to 12% of the total formulation, Commphora mukul CO₂ Extract in an amount 0.1 to 5% of the total formulation, Cinnamomum camphora CO₂ oil/steam distilled oil in an amount 0.1 to 20% of the total formulation, Celastrus paniculatus CO₂ oil/fixed oil in an amount 0.1 to 25% of the total formulation, Boswellia serrata steam distilled oil in an amount 0.1 to 25% of the total formulation, Vitex negundo CO₂ extract in an amount 0.1 to 5% of the total formulation, Bacopa monnieri CO₂ Extract in an amount 0.1 to 5% of the total formulation, Cyperus rotundus CO₂ Extract in an amount 0.1 to 5% of the total formulation, Centella asiatica CO₂ Extract in an amount 0.1 to 5% of the total formulation, Myristica fragrans CO₂ oil in an amount 0.1 to 30% of the total formulation, Valeriana Wallichii CO₂ oil in an amount 0.1 to 10% of the total formulation, Nardostachys jatamansi CO₂ oil in an amount 0.1 to 40% of the total formulation, for use in the treatment of Neurological diseases like ADHD, Alzheimer, Huntington, Parkinson, Epilepsy, CNS depression etc. and other disorders, like cough, cold, pain, immune system disorder, neurological disorders etc.

The fixed oils are selected from the group consisting of flaxseed oil, sesame seed oil, castor oil, sunflower oil, soyabean oil, safflower oil, corn oil, hemp oil, palm oil, and peanut oil, as a natural carrier in range of 60 to 98% of the total formulation.

In another preferred embodiment, all the herbal extracts and/or essential oils used in the formulations are extracted using supercritical CO₂ extraction with or without entrained solvent ethanol, using the below mentioned condition.

Pressure of extraction: Between 80 Kg/cm² to 350 Kg/cm²

Entrained ethanol: 0-10% (Preferred range 3-7%)

All the extracts and/or essential oils used are free from any hazardous hydrocarbon solvent residues such as hexane, chloroform, acetone etc., which are hazardous to human health when ingested even at very low residual levels. Such CO₂ extracted oils or extracts are combined together with one or more essential steam distilled oils such as Peppermint oil, Camphor oil, Basil oil, Boswellia oil, Cyperus oil or other essential oils from spices like Ginger, Sweet Orange oil, Cinnamon, Cardamom, Clove, Fennel, Anise seed, Cumin, etc.

These combinations are then mixed together with one or more fixed oils from plant material like flax seed oil, sesame seed oil, castor oil, sunflower oil, soya bean oil, safflower oil, corn oil, hemp oil, palm oil, peanut oil which are known to be safe for human consumption. They are proportioned in such a manner that the vaporization point of mixture is optimal in the range of 140° C. to 280° C. Use of such fixed oil eliminates use of chemical carriers like PEG, PG, Glycerin etc., which are known irritants and possible cause of hazardous effects due to use in E-cigarettes or other vaporizing devices used in the prior art.

Accordingly, the invention provides a method of formulating the therapeutic herbal formulation for vaporization and smoking which comprises:
a) combining CO₂ extracted oils or extracts of the plants selected in accordance with the invention with one or more essential steam distilled oils; and
b) mixing the step a) with one or more fixed oils from plant materials selected from the group consisting of flax seed oil sesame seed oil, castor oil, sunflower oil, soyabean oil, safflower oil, corn oil, hemp oil, palm oil, peanut oil in a proportion such that the vaporization point of mixture is optimal in the range of 140° C. to 280° C.

The one or more essential steam distilled oils are selected from the group consisting of Peppermint oil, Camphor oil, Basil oil, Boswellia oil, Cyperus oil or other essential oils from spices like Ginger, Sweet Orange oil, Cinnamon, Cardamom, Clove, Fennel, Anise seed, Cumin, etc.

Steam distilled oil are made using steam distillation and comprise of oils evaporated using steam. CO₂ extracted oils are made using CO₂ as a solvent and hence are different from steam distilled oils. There is no hydrolysis of any molecule in CO₂ extracted select oils unlike stream distilled essential oils. Therefore, molecular changes like hydrolysis does not happen in CO₂ extracted oils.

Accordingly, in a preferred embodiment, the invention provides a therapeutic herbal formulation comprising vaporizing blends consisting of one or more of herbal CO₂ extracts in an amount of 1 to 20%; one or more herbal CO₂ essential oils and/or essential steam distilled oils in an amount of 1 to 20% and together with one or more fixed oils in an amount of 60 to 98%, wherein, the composition is essentially devoid of chemical carriers viz., PEG, PG and Glycerin.

In a preferred embodiment, the essential oils are selected from the group consisting of Cinnamomum zeylanicum CO₂ oil in an amount 0.1 to 25% of the total formulation, Elettaria cardamomum oil in an amount 0.1 to 53% of the total formulation, Crocus sativus oil in an amount 0.1 to 11% of the total formulation, Santalum album steam distilled oil in an amount 0.1 to 20% of the total formulation, Ocimum sanctum CO₂ oil/steam distilled oil in an amount 0.1 to 35% of the total formulation, Curcuma longa CO₂ oil in an amount 0.1 to 30% of the total formulation, Zingiber officinale CO₂ oil/steam distilled oil in an amount 0.1 to 20% of the total formulation, Apium graveolens CO2 oil in an amount 0.1 to 20% of the total formulation, Mentha piperita oil in an amount 0.1 to 12% of the total formulation, Cinnamomum camphora CO₂ oil/steam distilled oil in an amount 0.1 to 20% of the total formulation, Celastrus paniculatus CO₂ oil/fixed oil in an amount 0.1 to 25% of the total formulation, Boswellia serrata steam distilled oil in an amount 0.1 to 25% of the total formulation, Myristica fragrans CO₂ oil in an amount 0.1 to 30% of the total formulation, Valeriana Wallichii CO₂ oil in an amount 0.1 to 10% of the total formulation, and Nardostachys jatamansi CO₂ oil in an amount 0.1 to 40% of the total formulation.

The herbal extracts are selected from the group consisting of Aquilaria agallocha CO₂ extract in an amount 0.1 to 1% of the total formulation, Withania somnifera CO₂ Extract in an amount 0.1 to 5% of the total formulation, Mucuna pruriens CO₂ Extract in an amount 0.1 to 5% of the total formulation, Emblica officinalis CO₂ Extract in an amount 0.1 to 2.0% of the total formulation, Terminalia chebula CO₂ Extract in an amount 0.1 to 2.0% of the total formulation, Terminalia bellerica CO₂ Extract in an amount 0.1 to 2.0% of the total formulation, Solanum xanthocarpum CO₂ Extract in an amount 0.1 to 1% of the total formulation, Glycyrrhiza glabra CO₂ Extract in an amount 0.1 to 1% of the total formulation, Commphora mukul CO₂ Extract in an amount 0.1 to 5% of the total formulation, Vitex negundo CO₂ extract in an amount 0.1 to 5% of the total formulation, Bacopa monnieri CO₂ Extract in an amount 0.1 to 5% of the total formulation, cyperus rotundus CO₂ Extract in an amount 0.1 to 5% of the total formulation, and Centella asiatica CO₂ Extract in an amount 0.1 to 5% of the total formulation.

The fixed oils are selected from the group consisting of flaxseed oil, sesame seed oil, castor oil, sunflower oil, soyabean oil, safflower oil, corn oil, hemp oil, palm oil, and peanut oil, as a natural carrier in range of 60 to 98% of the total formulation.

Therefore the present invention provides vaporizing blends of herbal extracts and/or essential oils that are designed with the intention of improved bioavailability with respect to various diseases and lesser side effects as compared to conventional oral administration of dosage form. The present invention uses mixtures of therapeutic extracts free from any hazardous hydrocarbon petrochemical based solvents. The present invention is also superior in terms of safety as it is made using 100% natural fixed oil/s as a carrier as against PG/PEG or Glycerin used in some of the prior arts which has known adverse health effects.

In another preferred embodiment, the present invention provides method of treating various disorders such as reducing/lowering/eliminating the symptoms of cough, cold, pain, immune system disorder, neurological disorders etc.; improving energy, relaxation, calmness and digestion, using the inventive compositions of herbal blends.

Additionally, the diseases which can be treated using the blends of the present invention are selected from Alzheimer, Huntington, Parkinson, Epilepsy, Seizures, alopecia including drug induced alopecia, Central Nervous system depression, gastrointestinal diseases such as irritable bowel syndrome, and upper respiratory tract infections etc. This drug delivery system provides direct drug delivery to the lungs to give faster onset of action and faster absorption.

In yet another embodiment, the formulations of the invention are provided with or without cannabinoids isolated from hemp plant which contains high amount of cannabinoids and low amount of THC which is psychoactive. Further, the above compositions may optionally contain CO₂ extract of hemp or marijuana plant by lowering the dosage of cannabinoids for getting desired therapeutic benefit.

Accordingly, in one preferred embodiment, the formulation comprises (1) Bacopa monnieri CO₂ Extract in an amount 1% of the total formulation, (2) Cyperus rotundus CO₂ Extract in an amount 2% of the total formulation, (3) Centella asiatica CO₂ Extract in an amount 1% of the total formulation, (4) Myristica fragrans CO₂ oil in an amount 20% of the total formulation, (5) Valeriana wallichii CO₂ oil in an amount 10% of the total formulation, (6) Nardostachys jatamansi CO₂ oil in an amount 39.33% of the total formulation, (7) Santalum album CO₂ oil in an amount 10% of the total formulation, (8) Celastrus paniculatus CO₂ oil in an amount 16.67% of the total formulation along with one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98%, for use in the treatment of Anxiety, insomnia or other stress related disorder etc.

In yet another embodiment, the invention provides a method of reducing/lowering/eliminating the symptoms of cough, cold, pain, energy, relaxation, calmness, digestion, immune system disorder, neurological disorders wherein, the method comprises administering a therapeutic herbal composition for smoking and vaporization provided in accordance with the invention, comprising one or more herbal extracts in an amount of 1 to 20%; one or more essential oils in an amount of 1 to 20% and together with one or more fixed oils in an amount of 60 to 98%, wherein, the composition is essentially devoid of chemical carriers viz., PEG, PG and Glycerin.

In a further embodiment, the invention provides use of therapeutic herbal composition for use in smoking and vaporization in accordance with the invention comprising one or more herbal extracts in an amount of 1 to 20%; one or more essential oils in an amount of 1 to 20% and together with one or more fixed oils in an amount of 60 to 98%, and wherein, the composition is essentially devoid of chemical carriers viz., PEG, PG and Glycerin.

In the traditional system of Dhumpan generally smoke was produced by using dried powder of herb, in which the powder was wrapped in the cotton, paper, leaves of the plants or it was directly burnt openly. However the smoke has several disadvantages which include formation of tar, toxic gases (i.e. Carbon monoxide, tar etc.) due to burning of all material in the fire or burning device/material used for such “Dhumpan”, which may cause respiratory disorders and may be carcinogenic to those exposed to first or second hand smoke.

All the disadvantages of traditional systems are minimized by present invention with the use of essential oil and/or CO₂ extracts of the herbs with fixed edible oils as carriers in which only desired substances are vaporized using high temperature air, thereby minimizing the hazards of traditional Dhumpan. Thus the present invention provides superior way of administrating medicated vapors without any adverse substances than that of traditional “Dhumpan ceremony” or “Medicated smoke”.

The following examples, which include preferred embodiments, will serve to illustrate the practice of this invention, it being understood that the particulars shown are by way of example and for purpose of illustrative discussion of preferred embodiments of the invention.

### EXAMPLES

### Example 1

A formulation for smoking and vaporization for use in the treatment of anxiety, mental stress and insomnia and to impart restful sleep

(1) Bacopa monnieri CO₂ Extract in an amount 0.1 to 0.3% of the total formulation, (2) cyperus rotundus CO₂ Extract in an amount 0.1 to 1% of the total formulation, (3) Centella asiatica CO₂ Extract in an amount 0.1 to 0.3% of the total formulation, (4) Myristica fragrans CO₂ oil in an amount 0.1 to 10% of the total formulation, (5) Valeriana Wallichii CO₂ oil in an amount 0.1 to 3% of the total formulation, (6) Nardostachys jatamansi CO₂ oil in an amount 0.1 to 10% of the total formulation, (7) Santalum album CO₂ oil in an amount 0.1 to 5% of the total formulation, (8) Celastrus paniculatus CO₂ oil in an amount 0.1 to 5% of the total formulation. (9) Rosmarinus officinalis CO2 extract in an amount 0.1 to 1% of the total formulation; (10) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

### Example 2

A formulation for smoking and vaporization for use in the treatment for improving energy, stamina, physical strength & secretions of pleasure inducing hormones and improving sexual health

(1) Cinnamomum zeylanicum CO₂ oil in an amount 0.1 to 7% of the total formulation, (2) Elettaria cardamomum CO₂ oil in an amount 0.1 to 14% of the total formulation, (3) Aquilaria agallocha CO₂ Extract in an amount 0.1 to 1% of the total formulation, (4) Crocus sativus CO₂ oil in an amount 0.1 to 3% of the total formulation, (5) Santalum album CO₂ oil in an amount 0.1 to 2.5% of the total formulation, (6) Withania somnifera CO₂ Extract an amount 0.1 to 0.5% of the total formulation, (7) Mucuna puriens CO₂ extract in an amount 0.1 to 0.5% of the total formulation, (8) Tribulus terrestris CO₂ Extract in an amount 0.1 to 0.5% of the total formulation, (9) Zingiber officinale CO₂ Extract in an amount 0.1 to 0.5% of the total formulation, (10) Asparagus racemosus CO₂ Extract in an amount 0.1 to 0.5% of the total formulation, (11) Rosmarinus officinalis CO₂ extract in an amount 0.1 to 1% of the total formulation and (12) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

### Example 3

A formulation for smoking and vaporization for use in the treatment to reduce joint pain muscular pain, COX-2 related pain, migraine, headache etc.

(1) Commphora mukul CO₂ oil in an amount 0.1 to 0.50% of the total formulation, (2) Curcuma longa CO₂ oil in an amount 0.1 to 5% of the total formulation, (3) Zingiber officinale P&N oil in an amount 0.1 to 5% of the total formulation, (4) Cinnamomum Camphora P&N oil in an amount 0.1 to 5% of the total formulation, (5) Cardiospermum halicacabum CO₂ oil in an amount 0.1 to 3% of the total formulation, (6) Boswellia Serrata P&N oil in an amount 0.1 to 5% of the total formulation, (7) Virtex Nirgundi CO₂ extract in an amount 0.1 to 0.5% of the total formulation, (8) Sweet Orange P&N Oil in an amount 0.1 to 4% of the total formulation. (9) Cinnamon CO₂ Extract in an amount 0.1 to 7% of the total formulation. (10) Sandalwood P&N Extract in an amount 0.1 to 1.5% of the total formulation, (11) Phyllanthus emblica CO₂ extract in an amount 0.1 to 0.30% of the total formulation. (12) Eclipta alba CO₂ oil in an amount 0.1 to 0.30% of the total formulation, (13) Rosmarinus officinalis CO₂ extract in an amount 0.1 to 1% of the total formulation, (14) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

### Example 4

A formulation for smoking and vaporization for use in the treatment to improve focus, short term memory, reducing symptoms and related ailments to ADHD, dementia, overall improvement in brain function and improve antioxidant effect.

(1) Curcuma longa CO₂ oil in an amount 0.1 to 15% of the total formulation, (2) Cardiospermum halicacabum CO₂ oil in an amount 0.1 to 8.3% of the total formulation, (3) Bacopa monnieri CO₂ oil in an amount 0.1 to 0.50% of the total formulation, (4) Nardostachys jatamansi P&N oil in an amount 0.1 to 2.40% of the total formulation, (5) Boswellia serrata P&N oil in an amount 0.1 to 4.20% of the total formulation, (6) Papermint P&N oil in an amount 0.1 to 4% of the total formulation, (7) Mentha spicata P&N oil in an amount 0.1 to 4% of the total formulation, (8) Sandal Wood P&N Oil in an amount 0.1 to 1.50% of the total formulation, (9) Rosmarinus officinalis CO₂ extract in an amount 0.1 to 1% of the total formulation, (10) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

### Example 5

A formulation for smoking and vaporization for use in the treatment for immune system disorders such as cancer, lupus, etc.

(1) Ocimum sanctum CO₂ oil in an amount 0.1 to 7% of the total formulation, (2) Hedychium spicatum CO₂ in an amount 0.1 to 5% of the total formulation, (3) Ocimum sanctum CO₂ Extract in an amount 0.1 to 3% of the total formulation, (4) Mentha spicata oil in an amount 0.1 to 7% of the total formulation, (5) Eclipta alba CO₂ in an amount 0.1 to 2.5% of the total formulation, (6) Cymbopegon citratus CO₂ Extract in an amount 0.1 to 6% of the total formulation, (7) Piper betle CO₂ oil in an amount 0.1 to 2% of the total formulation; (8) Curcuma longa CO₂ oil in an amount 0.1 to 4% of the total formulation; (9) Zingiber officinale CO₂ oil in an amount 0.1 to 4% of the total formulation; (10) Emblica officinalis CO₂ Extract in an amount 0.1 to 2% of the total formulation. (11) Tinospora cordifolia CO₂ extract in an amount 0.1 to 2% of the total formulation; (12) Santalum album in an amount 0.1 to 2% of the total formulation; (13) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

### Example 6

A formulation for smoking and vaporization for use in reducing cough and cold, Asthma, Coryza with other associated symptoms related to upper respiratory tract disorders

1) Emblica officinalis CO₂ in an amount 0.01 to 1% of the total formulation, (2) Terminalia chebula CO₂ in an amount 0.01 to 1% of the total formulation, (3) Terminalia belerica CO₂ Extract in an amount 0.01 to 1% of the total formulation, (4) Ringuni oil in an amount 0.1 to 2% of the total formulation, (5) Glycyrrhiza glabra CO₂ in an amount 0.1 to 3% of the total formulation, (6) Ocimum sanctum CO₂ Extract in an amount 0.1 to 4% of the total formulation, (7) Curcuma longa CO₂ oil in an amount 0.1 to 4% of the total formulation, (8) Zingiber officinale CO₂ oil in an amount 0.1 to 4% of the total formulation, (9) Ocimum sanctum CO₂ oil in an amount 0.1 to 10% of the total formulation, (10) Cinnamomum zeylanicum CO₂ oil Extract in an amount 0.1 to 4% of the total formulation, (11) Apium leptophyllum CO₂ oil in an amount 0.1 to 7% of the total formulation, (12) Mentha piperitain in an amount 0.1 to 4% of the total formulation, (13) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

### Example 7

A formulation for smoking and vaporization for use in the treatment for improving digestion and supporting digestive system, heat burn, greed, irritable bowel syndrome

(1) Emblica officinalis CO₂ Extract in an amount 0.1 to 0.3% of the total formulation, (2) Terminalia chebula CO₂ extract in an amount 0.05 to 0.09% of the total formulation, (3) Terminalia bellerica CO₂ Extract in an amount 0.05 to 0.09% of the total formulation, (4) Asparagus racemosus CO₂ oil in an amount 0.01 to 0.05% of the total formulation, (5) Tinospora cordifolia in an amount 0.05 to 0.09% of the total formulation, (6) Piper betle CO₂ oil in an amount 0.1 to 0.5% of the total formulation, (7) Foeniculum vulgare CO₂ oil in an amount 18 to 20% of the total formulation, (8) Mentha spicata CO₂ Extract in an amount 1 to 3% of the total formulation. (9) Trachyspermum ammi CO₂ oil in an amount 1 to 3% of the total formulation (10) Rosmarinus officinalis CO₂ extract in an amount 0.1 to 1% of the total formulation, (11) one or more fixed oils selected from the group consisting of flax seed oil, sesame seed oil, castor oil, sunflower oil, soyabean oil, safflower oil, corn oil, hemp oil, palm oil, and peanut oil, as a natural carrier in range of 60 to 98% of the total formulation.

### Example 8

A formulation for smoking and vaporization for use in the treatment for Epilepsy & seizers

(1) Azadirachta indica leaf CO₂ Extract in an amount 0.1 to 0.3% of the total formulation, (2) Celastrus paniculatus CO₂ extract in an amount 8 to 12% of the total formulation, (3) Trachyspermum ammi CO₂ Extract in an amount 3 to 5% of the total formulation, (4) Boswellia serrate CO₂ oil in an amount 6 to 8% of the total formulation, (5) Pandanus odorifer oil in an amount 0.1 to 3% of the total formulation, (6) Bacopa monnieri CO₂ Extract in an amount 0.1 to 0.5% of the total formulation, (7) Rosmarinus officinalis CO₂ extract in an amount 0.1 to 1% of the total formulation, (8) one or more fixed oils selected from the group consisting of flax seed oil, sesame seed oil, castor oil, sunflower oil, soyabean oil, safflower oil, corn oil, hemp oil, palm oil, and peanut oil, as a natural carrier in range of 60 to 98% of the total formulation.

### Example 9

A formulation for smoking and vaporization for use in the treatment for neuron regeneration and increase hippocampus by antioxidants and anti-inflammatory activity.

(1) Curcuma longa CO₂ oil containing 40 to 55% turmerones in an amount 5 to 40% of the total formulation, (2) Celastru spaniculatus CO₂ extract or oil in an amount 0.1 to 10% of the total formulation, (3) Rosmarinus officinalis CO₂ extract in an amount 0.1 to 1% of the total formulation and also CBD and THC from Canabis Sativa or Canabis indica, (4) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation with minimum omega 3 fatty acids (EPA) 10 to 65% of total formulation.

### Example 10

A formulation for smoking and vaporization for use in the treatment for hair growth in case of Alopecia or drug induced Alopecia.

(1) Eclipta alba CO₂ extract in an amount 0.1 to 10% of the total formulation, (2) Trigonella foenum-graecum CO₂ oil in an amount 0.1 to 1% of the total formulation, (3) Tribulus terrestris CO₂ Extract in an amount 0.1 to 1% of the total formulation, (4) Centella asiatica CO₂ Extract in an amount 0.1 to 1% of the total formulation, (5) Emblica officinalis CO₂ Extract in an amount 0.1 to 1% of the total formulation, (6) Hedychium spicatum CO₂ Extract in an amount 0.1 to 1% of the total formulation, (7) Acacia concinna CO₂ Extract in an amount 0.1 to 1% of the total formulation, (8) Santalum album CO₂ oil in an amount 0.1 to 2.5% of the total formulation, (9) Rosmarinus officinalis CO₂ extract in an amount 0.1 to 1% of the total formulation, (10) one or more fixed oils are selected from the group consisting of flax seed oil, sesame seed oil, castor oil, sunflower oil, soyabean oil, safflower oil, corn oil, hemp oil, palm oil, and peanut oil, as a natural carrier in range of 60 to 98% of the of the total formulation.

We claim:

1. A therapeutic herbal composition for smoking and vaporization comprising one or more herbal CO₂ extracts in an amount of 1 to 20%; one or more CO₂ essential oils and/or essential steam distilled oils in an amount of 1 to 20% together with one or more fixed oils selected from the group consisting of flax seed oil, sesame seed oil, castor oil, sunflower oil, soyabean oil, safflower oil, corn oil, hemp oil, palm oil, and peanut oil, in an amount of 60 to 98%, wherein, the composition is essentially devoid of chemical carriers viz., PEG, PG and Glycerin, for use in treating different disease conditions like cough, cold, pain, energy, relaxation, calmness, digestion, immune system disorder and neurological disorders.

2. The therapeutic herbal composition for smoking and vaporization according to claim 1, wherein the one or more essential oils are selected from the group consisting of Cinnamomum zeylanicum CO₂ oil in an amount 0.1 to 25% of the total formulation, Elettaria cardamomum oil in an amount 0.1 to 53% of the total formulation, Crocus sativus oil in an amount 0.1 to 11% of the total formulation, Santalum album steam distilled oil in an amount 0.1 to 20% of the total formulation, Ocimum sanctum CO₂ oil/steam distilled oil in an amount 0.1 to 35% of the total formulation, Curcuma longa CO₂ oil in an amount 0.1 to 30% of the total formulation, Zingiber officinale CO₂ oil/steam distilled oil in an amount 0.1 to 20% of the total formulation, Apium graveolens CO₂ oil in an amount 0.1 to 20% of the total formulation, Mentha piperita oil in an amount 0.1 to 12% of the total formulation, Cinnamomum camphora CO₂ oil/steam distilled oil in an amount 0.1 to 20% of the total formulation, Celastrus paniculatus CO₂ oil/fixed oil in an amount 0.1 to 25% of the total formulation, Boswellia serrata steam distilled oil in an amount 0.1 to 25% of the total formulation, Myristica fragrans CO₂ oil in an amount 0.1 to 30% of the total formulation, Valeriana Wallichii CO₂ oil in an amount 0.1 to 10% of the total formulation, and Nardostachys jatamansi CO₂ oil in an amount 0.1 to 40% of the total formulation.

3. The therapeutic herbal composition for smoking and vaporization according to claim 1, wherein the one or more herbal extracts are selected from the group consisting of Aquilaria agallocha CO₂ extract in an amount 0.1 to 1% of the total formulation, Withania somnifera CO₂ Extract in an amount 0.1 to 5% of the total formulation, Mucuna pruriens CO₂ Extract in an amount 0.1 to 5% of the total formulation, Emblica officinalis CO₂ Extract in an amount 0.1 to 2.0% of the total formulation, Terminalia chebula CO₂ Extract in an amount 0.1 to 2.0% of the total formulation, Terminalia bellerica CO₂ Extract in an amount 0.1 to 2.0% of the total formulation, Solanum xanthocarpum CO₂ Extract in an amount 0.1 to 1% of the total formulation, Glycyrrhiza glabra CO₂ Extract in an amount 0.1 to 1% of the total formulation, Commphora mukul CO₂ Extract in an amount 0.1 to 5% of the total formulation, Vitex negundo CO₂ extract in an amount 0.1 to 5% of the total formulation, Bacopa monnieri CO₂ Extract in an amount 0.1 to 5% of the total formulation, Cyperus rotundus CO₂ Extract in an amount 0.1 to 5% of the total formulation, and Centella asiatica CO₂ Extract in an amount 0.1 to 5% of the total formulation.

4. The therapeutic composition for smoking and vaporization according to claim 1, optionally comprises CO₂ extract of hemp or marijuana plant with low dosage of cannabinoids.

5. The therapeutic herbal composition for smoking and vaporization according to claim 1, for use in the treatment of anxiety, mental stress and insomnia and to impart restful sleep, said composition comprising;
a) Bacopa monnieri CO₂ Extract in an amount 0.1 to 0.3% of the total formulation;
b) cyperus rotundus CO₂ Extract in an amount 0.1 to 1% of the total formulation;
c) Centella asiatica CO₂ Extract in an amount 0.1 to 0.3% of the total formulation;
d) Myristica fragrans CO₂ oil in an amount 0.1 to 10% of the total formulation;
e) Valeriana Wallichii CO₂ oil in an amount 0.1 to 3% of the total formulation;
f) Nardostachys jatamansi CO₂ oil in an amount 0.1 to 10% of the total formulation;
g) Santalum album CO₂ oil in an amount 0.1 to 5% of the total formulation;
h) Celastrus paniculatus CO₂ oil in an amount 0.1 to 5% of the total formulation;
i) Rosmarinus officinalis CO₂ extract in an amount 0.1 to 1% of the total formulation;
j) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

6. The therapeutic herbal composition for smoking and vaporization according to claim 1, for use in the treatment for improving energy, stamina, physical strength & secretions of pleasure inducing hormones and improving sexual health, comprising;
a) Cinnamomum zeylanicum CO₂ oil in an amount 0.1 to 7% of the total formulation;
b) Elettaria cardamomum CO₂ oil in an amount 0.1 to 14% of the total formulation;
c) Aquilaria agallocha CO₂ extract in an amount 0.1 to 1% of the total formulation;
d) Crocus sativus CO₂ oil in an amount 0.1 to 3% of the total formulation;
e) Santalum album CO₂ oil in an amount 0.1 to 2.5% of the total formulation;
f) Withania somnifera CO₂ extract in an amount 0.1 to 0.5% of the total formulation;
g) Mucuna puriens CO₂ extract in an amount 0.1 to 0.5% of the total formulation;
(h) Tribulus terrestris CO₂ extract in an amount 0.1 to 0.5% of the total formulation;
i) Zingiber officinale CO₂ extract in an amount 0.1 to 0.5% of the total formulation;
j) Asparagus racemosus CO₂ extract in an amount 0.1 to 0.5% of the total formulation;
k) Rosmarinus officinalis CO₂ extract in an amount 0.1 to 1% of the total formulation and (12) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

7. The therapeutic herbal composition for smoking and vaporization according to claim 1, for use in the treatment to reduce joint pain muscular pain, COX-2 related pain, migraine, headache, comprising
a) Commphora mukul CO₂ oil in an amount 0.1 to 0.50% of the total formulation;
b) Curcuma longa CO₂ oil in an amount 0.1 to 5% of the total formulation;
c) Zingiber officinale P&N oil in an amount 0.1 to 5% of the total formulation;
d) Cinnamomum camphora P&N oil in an amount 0.1 to 5% of the total formulation;
e) Cardiospermum halicacabum CO₂ oil in an amount 0.1 to 3% of the total formulation;
f) Boswellia serrata P&N oil in an amount 0.1 to 5% of the total formulation;
g) Virtex nirgundi CO₂ extract in an amount 0.1 to 0.5% of the total formulation;
h) Sweet Orange P&N Oil in an amount 0.1 to 4% of the total formulation;
i) Cinnamon CO₂ extract in an amount 0.1 to 7% of the total formulation;
j) Sandal Wood P&N extract in an amount 0.1 to 1.5% of the total formulation;
k) Phyllanthus Emblica CO₂ extract in an amount 0.1 to 0.30% of the total formulation;
l) Eclipta alba CO₂ oil in an amount 0.1 to 0.30 of the total formulation;
m) Rosmarinus officinalis CO₂ extract in an amount 0.1 to 1% of the total formulation; and
n) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

8. The therapeutic herbal composition for smoking and vaporization according to claim 1, for use in the treatment to improve focus, short term memory, reducing symptoms and related ailments to ADHD, dementia, overall improvement in brain function and improve antioxidant effect, comprising:
a) Curcuma longa CO₂ oil in an amount 0.1 to 15% of the total formulation;
b) Cardiospermum halicacabum CO₂ oil in an amount 0.1 to 8.3% of the total formulation;
c) Bacopa monnieri CO₂ oil in an amount 0.1 to 0.50% of the total formulation;
d) Nardostachys jatamansi P&N oil in an amount 0.1 to 2.40% of the total formulation;
e) Boswellia serrata P&N oil in an amount 0.1 to 4.20% of the total formulation;
f) Papermint P&N oil in an amount 0.1 to 4% of the total formulation;
g) Mentha P&N oil in an amount 0.1 to 4% of the total formulation;
h) Sandal Wood P&N Oil in an amount 0.1 to 1.50% of the total formulation;
i) Rosmarinus officinalis CO₂ extract in an amount 0.1 to 1% of the total formulation; and
j) one or more fixed oils selected from the group consisting of flax seed oil, sesame seed oil, castor oil, sunflower oil, soyabean oil, safflower oil, corn oil, hemp oil, palm oil, and peanut oil, as a natural carrier in range of 60 to 98% of the total formulation.

9. The therapeutic herbal composition for smoking and vaporization according to claim 1, for use in the treatment for immune system disorders such as cancer, lupus, comprising;
a) Ocimum sanctum CO₂ oil in an amount 0.1 to 7% of the total formulation;
b) Hedychium spicatum CO₂ in an amount 0.1 to 5% of the total formulation;
c) Ocimum sanctum CO₂ extract in an amount 0.1 to 3% of the total formulation;
d) Mentha spicata oil in an amount 0.1 to 7% of the total formulation;
e) Eclipta alba CO₂ in an amount 0.1 to 2.5% of the total formulation;
f) Cymbopegon citratus CO₂ extract in an amount 0.1 to 6% of the total formulation;
g) Piper betle CO₂ oil in an amount 0.1 to 2% of the total formulation;
h) Curcuma longa CO₂ oil in an amount 0.1 to 4% of the total formulation;
i) Zingiber officinale CO₂ oil in an amount 0.1 to 4% of the total formulation;
j) Emblica officinalis CO₂ extract in an amount 0.1 to 2% of the total formulation;
k) Tinospora cordifolia CO₂ extract in an amount 0.1 to 2% of the total formulation;
l) Santalum album oil in an amount 0.1 to 2% of the total formulation; and
m) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

10. The therapeutic herbal composition for smoking and vaporization according to claim 1, for use in reducing cough and cold, Asthma, Coryza with other associated symptoms related to upper respiratory tract disorders comprising;
a) Emblica officinalis CO₂ in an amount 0.01 to 1% of the total formulation;
b) Terminalia chebula CO₂ in an amount 0.01 to 1% of the total formulation;
c) Terminalia belerica CO₂ Extract in an amount 0.01 to 1% of the total formulation;
d) Ringuni oil in an amount 0.1 to 2% of the total formulation;
e) Glycyrrhiza glabra CO₂ in an amount 0.1 to 3% of the total formulation;
f) Ocimum sanctum CO₂ extract in an amount 0.1 to 4% of the total formulation;
g) Curcuma longa CO₂ oil in an amount 0.1 to 4% of the total formulation;
h) Zingiber officinale CO₂ oil in an amount 0.1 to 4% of the total formulation;
i) Ocimum sanctum CO₂ oil in an amount 0.1 to 10% of the total formulation;
j) Cinnamomum zeylanicum CO₂ oil Extract in an amount 0.1 to 4% of the total formulation;
k) Apium leptophyllum CO₂ oil in an amount 0.1 to 7% of the total formulation;
l) Mentha piperita in an amount 0.1 to 4% of the total formulation; and
m) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

11. The therapeutic herbal composition for smoking and vaporization according to claim 1, for use in the treatment for improving digestion and supporting digestive system, heat burn, greed, irritable bowel syndrome comprising;
a) Emblica officinalis CO₂ Extract in an amount 0.1 to 0.3% of the total formulation;
b) Terminalia chebula CO₂ extract in an amount 0.05 to 0.09% of the total formulation;
c) Terminalia Billerica CO₂ extract in an amount 0.05 to 0.09% of the total formulation;
d) Asparagus racemosus CO₂ oil in an amount 0.01 to 0.05% of the total formulation;
e) Tinospora cordifolia in an amount 0.05 to 0.09% of the total formulation;
f) Piper betle CO₂ oil in an amount 0.1 to 0.5% of the total formulation;
g) Foeniculum vulgare CO₂ oil in an amount 18 to 20% of the total formulation;
h) Mentha spicata CO₂ Extract in an amount 1 to 3% of the total formulation;
i) Trachypermum ammi CO₂ oil in an amount 1 to 3% of the total formulation;
j) Rosmarinu sofficinalis CO₂ extract in an amount 0.1 to 1% of the total formulation; and
k) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

12. The therapeutic herbal composition for smoking and vaporization according to claim 1, for use in the treatment of Epilepsy & seizers comprising;
a) Azadirachta indica leaf CO₂ Extract in an amount 0.1 to 0.3% of the total formulation;
b) Celastrus paniculatus CO₂ extract in an amount 8 to 12% of the total formulation;
c) Trachypermum ammi CO₂ extract in an amount 3 to 5% of the total formulation;
d) Boswellia serrata CO₂ oil in an amount 6 to 8% of the total formulation;
e) Pandanus odorifer oil in an amount 0.1 to 3% of the total formulation;
Bacopa monnieri CO₂ extract in an amount 0.1 to 0.5% of the total formulation;
g) Rosmarinus officinalis CO₂ extract in an amount 0.1 to 1% of the total formulation; and
h) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation.

13. The therapeutic herbal composition for smoking and vaporization according to claim 1, for use in the treatment of neuron regeneration and increase hippocampus by antioxidants and anti-inflammatory activity. comprising;
a) Curcuma longa CO₂ oil containing 40 to 55% turmerones in an amount 5 to 40% of the total formulation,
b) Celastrus paniculatus CO₂ extract or oil in an amount 0.1 to 10% of the total formulation,
c) Rosmarinus officinalis CO₂ extract in an amount 0.1 to 1% of the total formulation and also CBD and THC from Canabis Sativa or Canabis indica plant parts
d) one or more fixed oils selected from the group consisting of flax seed, sesame seed oil, castor oil, sunflower oil, soyabean oil, safflower oil, corn oil, hemp oil, palm oil, and peanut oil, as a natural carrier in range of 60 to 98% of the total formulation with minimum omega 3 fatty acids (EPA) 10 to 65% of total formulation.

14. The therapeutic herbal composition for smoking and vaporization according to claim 1, for use in the treatment of for hair growth in case of Alopecia or drug induced Alopecia comprising;
a) Eclipta alba CO₂ extract in an amount 0.1 to 10% of the total formulation;
b) Trigonella foenum-graecum CO₂ oil in an amount 0.1 to 1% of the total formulation;
c) Tribulus terrestris CO₂ extract in an amount 0.1 to 1% of the total formulation;
d) Centella asiatica CO₂ extract in an amount 0.1 to 1% of the total formulation;
e) Emblica officinalis CO₂ extract in an amount 0.1 to 1% of the total formulation;
f) Hedychium spicatum CO₂ extract in an amount 0.1 to 1% of the total formulation;
g) Acacia concinna CO₂ extract in an amount 0.1 to 1% of the total formulation;
h) Santalum album oil in an amount 0.1 to 2.5% of the total formulation;
i) Rosmarinus officinalis CO₂ extract in an amount 0.1 to 1% of the total formulation; and
j) one or more fixed oils are selected from the group consisting of flax seed, sesame seed oil, castor oil, sunflower oil, soyabean oil, safflower oil, corn oil, hemp oil, palm oil, and peanut oil, as a natural carrier in range of 60 to 98% of the of the total formulation.

15. A method of formulating a therapeutic herbal formulation for vaporization and smoking which comprises:
a) combining a CO₂ extracted oil or extract with one or more essential steam distilled oils; and
b) mixing the product of step a) with one or more fixed oils from plant material like flax seed oil, sesame seed oil, castor oil, sunflower oil, soyabean oil, safflower oil, corn oil, hemp oil, palm oil, peanut oil in a proportion such that the vaporization point of mixture is optimal in the range of 140° C. to 280° C.

16. A method for reducing/lowering/eliminating the symptoms of cough, cold, pain, and improving the energy, relaxation, calmness, digestion, immune system disorder, neurological disorders wherein, the method comprises administering a therapeutic herbal composition for smoking and vaporization in accordance with claim 1, comprising one or more herbal extracts in an amount of 1 to 20%; one or more essential oils and/or steam distilled oils in an amount of 1 to 20% and optionally together with one or more fixed oils selected from the group consisting of flax seed oil, sesame seed oil, castor oil, sunflower oil, soyabean oil, safflower oil, corn oil, hemp oil, palm oil, and peanut oil, in an amount of 60 to 98%, wherein, the composition is essentially devoid of chemical carriers viz., PEG, PG and Glycerin.

17. The method according to claim 15, wherein, the herbal extracts are selected from the group consisting of Aquilaria agallocha CO₂ extract, Withania somnifera CO₂ Extract, Mucuna pruriens CO₂ Extract, Emblica officinalis CO₂ Extract, Terminalia chebula CO₂ Extract, Terminalia bellerica CO₂ Extract, Solanum xanthocarpum CO₂ Extract, Glycyrrhiza glabra CO₂ Extract, Commiphora mukul CO₂ Extract, Vitex negundo CO₂ extract, Bacopa monnieri CO₂ Extract, Cyperus rotundus CO₂ Extract, and Centella asiatica CO₂ Extract.

18. The method according to claim 15, wherein, the essential oils are selected from the group consisting of Cinnamomum zeylanicum CO₂ oil, Elettatia cardamomum oil, Crocus sativus oil, Santalum album steam distilled oil, Ocimum sanctum CO₂ oil/steam distilled oil, Curcuma longa CO₂ oil, Zingiber officinale CO₂ oil/steam distilled oil, Apium graveolens CO₂ oil, Mentha piperita oil, Cinnamomum camphora CO₂ oil/steam distilled oil, Celastrus paniculatus CO₂ oil/fixed oil, Boswellia serrata steam distilled oil, Myristica fragrans CO₂ oil, Valeriana Wallichii CO₂ oil, and Nardostachys jatamansi CO₂ oil.
